(19) United States
(12) Patent Application Publication
(10) Pub. No.: US 2024/0108260 A1
(43) Pub. Date: Apr. 4, 2024

(54) System For Managing Treatment Of A Patient By Measuring Emotional Response

(71) Applicant: GAIA AG, Hamburg (DE)

(72) Inventor: Mario Weiss, Hamburg (DE)

(21) Appl. No.: 17/937,265

(22) Filed: Sep. 30, 2022

(51) Int. Cl. A61B 5/16; A61B 5/00; G16H 20/00

(52) U.S. Cl. CPC A61B 5/165; A61B 5/0077; G16H 20/00

(57) ABSTRACT

A system for managing treatment of a patient is provided with a computer, a database in data communication with said computer, said database having a plurality of prompts, a user device in data communication with said computer for presenting a first prompt received from said computer to the patient, a camera associated with said user device for taking an image of the patient's face in response to the prompt, software executing on said computer for receiving the image and determining an emotion associated with said image, software executing on said user device which may present the determined emotion to the patient, software executing on said computer which may receive a confirmation as to whether the determined emotion is correct, and software executing on said computer querying the database based on the confirmed emotion to retrieve at least one additional prompts to be displayed to the patient to refine their treatment.

### TECHNICAL FIELD

The present disclosure relates to a system for determining the emotional state of a patient, and then using the emotional state to confirm or modify the patient's treatment. More specifically, the present disclosure relates to using cameras and a neural network to analyze the emotional state of a patient and refine a patient's pharmaceutical treatment based their determined emotional.

### BACKGROUND

Patients like to be understood by their caregivers. Feeling understood is also an important prerequisite for trust and bonding. Such trust and bonding are critical for the patient to follow therapeutic advice.

Physicians rarely have the time required to connect with patients on a deep emotional level. Often, they are too tired to express the proper level of clinical empathy and understanding. Even if they could, physicians are not available for the round-the-clock support that could be required for emotional crisis and instability care.

In addition, emotional expressions can vary wildly between individuals. Specific groups of patients, such as depressed patients, also may show a different reaction patterns to pharmaceutical treatment such that continuous evaluation would improve their care. In addition to time and cost restraints, physicians cannot be trained on every presentation of emotional expression made by various patient groups.

However, emotional support is very important for patients, especially those who are depressed, suffer from severe diseases or are in treatment for drug misuse.

### SUMMARY

For these and other reasons known to a person of an ordinary skill in the art, what is needed is a system that allows patients to be emotionally understood.

A goal of the present disclosure is to provide a system that accurately determines a patient's emotional state.

Another goal of the present disclosure is to use the determined emotional state to refine a patient's treatment.

Another goal of the present disclosure is to provide a system that verifies that the determined emotional state of a patient is correct.

Another goal of the present disclosure is to provide a system that can adequately react to cognitive and emotional signals of patients.

Another goal of the present disclosure is to provide a system that analyzes the facial expressions of patients to determine the patient's emotional state.

Another goal of the present disclosure is to provide a system that can provide continuous or near-continuous monitoring a patient's emotional state.

Another goal of the present disclosure is to provide a system that can receive feedback to better understand emotional states.

Another goal of the present disclosure is to provide a system that can signal third parties in an emergency.

In one aspect of the present invention, a system for managing treatment of a patient is provided with a computer and a database in data communication with said computer. The database has a plurality of prompts. A user device is also in data communication with said computer for presenting a first prompt received from said computer to the patient. A camera associated with said user device takes an image of the patient's face in response to the prompt. Software executing on said computer for receives the image and determining an emotion associated with said image. Software executing on said user device presents the determined emotion to the patient. Software executing on said computer receives a confirmation as to whether the determined emotion is correct. Software executing on said computer queries the database based on the confirmed emotion to retrieve at least one additional prompts to be displayed to the patient to refine their treatment.

### BRIEF DESCRIPTION OF THE DRAWINGS

FIG. 1 shows a schematic diagram of the presently disclosed system.

### DETAILED DESCRIPTION

The present invention will now be described by referencing the appended FIGURE.

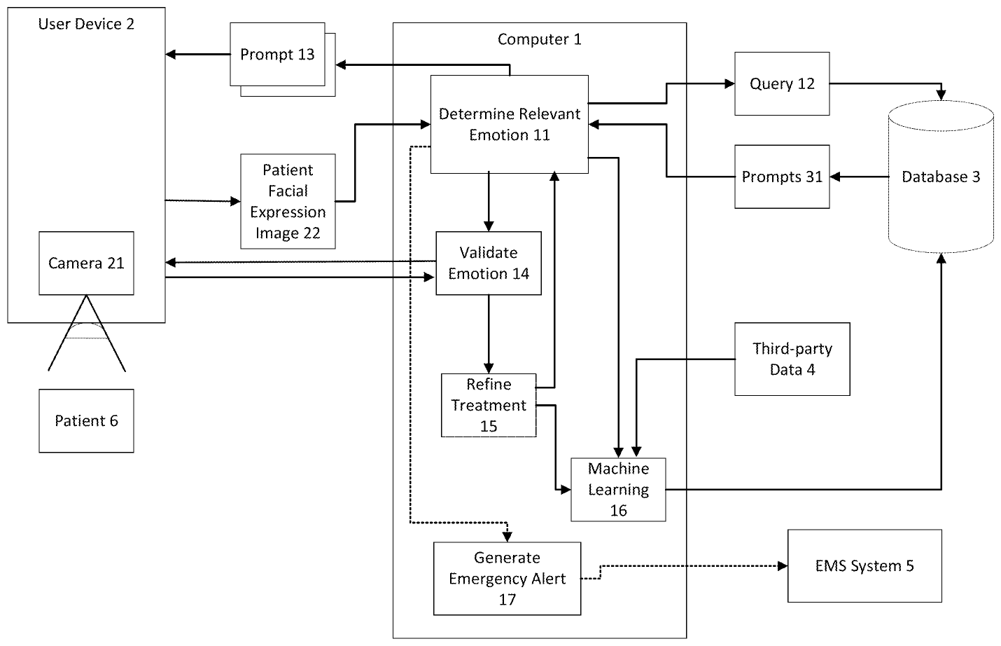

Referring to FIG. 1, the present disclosure describes a system 10 for emotional computing.

The system 10 includes a computer 1. The computer 1 may be a processor, remote computer, computer server, network, or any other computing resource, including mobile devices.

The computer 1 may be in data communication with a user device 2. The user device 2 may be a computer, laptop, smartphone, tablet, or other electronic device, including mobile devices, capable of transmitting data to the computer 1. User device 2 may run an application on a mobile device or smartphone. The user device 2 may have an input device such as a mouse and keyboard, touchscreen, trackpad, etc. The user device 2 may include a display. The user device 2 may include a camera 21. The camera 21 may be a webcam, still camera, video camera, etc. The camera 21 may be integrated in or external to the user device 2.

The computer 1 may also be in communication with a database 3. The database 3 may store information regarding the system 10, including queries 12 and prompts 31. The database 3 may be a storage drive or array accessible to computer 1, or cloud storage. Prompts 31 may be indexed or searchable by queries 12.

Prompts 31 may include any media that stimulates the patient 6. For example, prompts 31 may include text, images, sound, video, physical stimuli, tasting material, etc.

Queries 12 may seek a category of prompts 31, or a specific prompt 31. Categories of prompts 12 may on the prompt itself, the type of prompt (image, sound, etc.), previous prompts or the patient's reaction thereto, or categorizations based on conditions, medications, or other factors.

The computer 1 may include software 11 to determine the relevant emotion that a patient is experiencing. To that end, it may send a query 12 to the database 3 for a prompt 31. The prompt 31 may be sent to the user device 2 as prompt 13 after being processed by the software 11. The software 11 may process the prompt 31 by identifying an expected emotion in response to the prompt 31.

The user device 2 presents the prompt 13 to the patient 6. The patient 6 may experience a response in view of being presented with prompt 13. For instance, the patient's 6 facial expression may change. The camera 21 may capture images the patient's 6 facial expressions before, during, and after the patient is presented with the prompt 13.

The software 11 determines the relevant emotion based on the images captured by the camera 21. The software 11 may use specialized software to analyze the images captured by the camera 21. For example, computer vision software may be used to recognize facial expressions, and a neural network may be used to and classify the facial expressions as showing emotions. The software 11 may assign numeric values to emotions that may be shown in the images.

The computer may use third-party data 4 to help determine the relevant emotion 11. Third party data 4 may include the weather at location of patient 6, potential stressors such as crime rate (communicated crime in media), pollution, traffic (time spend in traffic), and psycho economics such as stock price, inflation rate, employment rate (specifically in the sector patient 6 is working in), and consumer index. For example, the system may weigh emotions differently if it is a sunny versus a rainy day, or if stocks are up or down.

The computer 1 may run software 14 to validate the determined emotion. For example, the computer 1 may display emotional information (text, picture, audio, video, etc.) that signals to the patient 6 that her emotions are well understood. For example, the computer may ask the patient 6 if they are “feeling a bit sad right now?”

In response to being asked to validate the determined emotion, the computer 1 may receive a reaction from the patient 6. The reaction may be in text, audio, video, or other form. For instance, the response may be a second series of images from the camera 21 taken during the emotional validation step. The computer 1 may run software 11 to determine the relevant emotion of this second series of images.

The patient 6 reaction may be used to identify whether the determined emotion is accurate. If the determined emotion was inaccurate, the computer 1 may present a new prompt 13.

If the determined emotion was accurate, the computer 1 refine treatment 15 based on the determined emotion. Refining treatment 15 may include adjusting prescriptions, initiating new prescriptions, performing physical therapy, attending counseling, or providing any other known treatments.

The computer may use any of the prompts 13, facial expressions 22, the results of the determine relevant emotion software 11 and validate emotion software 14, and third-party data 4 to perform machine learning 16 and improve its functionality. For example, the computer 1 may refine the determine relevant emotion software 11 using this information. Database 3 may store the results of the machine learning process 16. In this way, the system can learn from its continued use.

Other sensors may be used to gauge a patient's 6 response to a prompt 13. For example, microphones, heart rate sensors, blood pressure sensors, temperature sensors, and others may be used to measure a patient's 6 response to a prompt 13. Alternatively, the patient 6 may be asked to respond to the prompt 13 with words, either via text or spoken. The patient 6 may be asked to respond to the prompt 13 by picking an emotion, or a representation of an emotion (such as a heart for love). The software 11 may be configured to work with any type of these, or other, input methods.

The system may run in the background and not interfere with other treatments or activities. In such situations, the system may constantly refine its determination of the emotional state of the patient 6. The patient 6 may be aware of the system's determination and may choose to share or advertise their emotional state, such as on social media.

If a determined relevant emotion 11 is potentially harmful, the computer 1 may generate an emergency alert 17. The alert may be communicated to an EMS system 5, a designated contract, medical professional, or other person.

Although the invention has been illustrated and described herein with reference to a preferred embodiment and a specific example thereof, it will be readily apparent to those of ordinary skill that the art that other embodiments and examples may perform similar functions and/or achieve user experiences. All such equivalent embodiments and examples are within the spirit and scope of the present invention, are contemplated thereby, and are intended to be covered by the following claims.

In compliance with the statute, the present teachings have been described in language more or less specific as to structural and methodical features. It is to be understood, however, that the present teachings are not limited to the specific features shown and described, since the systems and methods herein disclosed comprise preferred forms of putting the present teachings into effect. The present disclosure is to be considered as an example of the invention, and is not intended to limit the invention to a specific embodiment illustrated by the FIGURES above or description below.

For purposes of explanation and not limitation, specific details are set forth such as particular architectures, interfaces, techniques, etc. in order to provide a thorough understanding. In other instances, detailed descriptions of well-known devices, circuits, and methods are omitted so as not to obscure the description with unnecessary detail.

Generally, all terms used in the claims are to be interpreted according to their ordinary meaning in the technical field, unless explicitly defined otherwise herein. All references to a/an/the element, apparatus, component, means, step, etc. are to be interpreted openly as referring to at least one instance of the element, apparatus, component, means, step, etc., unless explicitly stated otherwise. The steps of any method disclosed herein do not have to be performed in the exact order disclosed, unless explicitly stated. The use of “first”, “second,” etc. for different features/components of the present disclosure are only intended to distinguish the features/components from other similar features/components and not to impart any order or hierarchy to the features/components. As used herein, the term “and/or” includes any and all combinations of one or more of the associated listed items. As used herein, the term “application” is intended to be interchangeable with the term “invention”, unless context clearly indicates otherwise.

To aid the Patent Office and any readers of any patent issued on this application in interpreting the claims appended hereto, Applicant that it does not intend any of the claims or claim elements to invoke 35 U.S.C. 112(f) unless the words “means for” or “step for” are explicitly used in the particular claim.

While the present teachings have been described above in terms of specific embodiments, it is to be understood that they are not limited to these disclosed embodiments. Many modifications and other embodiments will come to mind to those skilled in the art to which this pertains, and which are intended to be and are covered by both this disclosure and the appended claims. It is intended that the scope of the present teachings should be determined by proper interpretation and construction of the appended claims and their legal equivalents, as understood by those of skill in the art relying upon the disclosure in this specification and the attached drawings. In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

What is claimed is:

1. A system for managing treatment of a patient comprising:
a computer;
a database in data communication with said computer, said database having a plurality of prompts;
a user device in data communication with said computer for presenting a first prompt received from said computer to the patient;
a camera associated with said user device for taking an image of the patient's face in response to the prompt;
software executing on said computer for receiving the image and determining an emotion associated with said image;
software executing on said user device for presenting the determined emotion to the patient;
software executing on said computer for receiving a confirmation as to whether the determined emotion is correct; and
software executing on said computer querying the database based on the confirmed emotion to retrieve at least one additional prompts to be displayed to the patient to refine their treatment.

2. The system of claim 1, further comprising software executing on said computer for generating an emergency alert in response to the patient confirmation.

3. The system of claim 1, further comprising software executing on said computer for training a machine learning algorithm based on at least one of the determined emotion and confirmation.

4. The system of claim 3, wherein the machine learning algorithm is the software determining an emotion associated with said image.

5. The system of claim 1, further comprising a determined refinement to the treatment.

6. The system of claim 5, further comprising software executing on said computer for training a machine learning algorithm based on at least one of the determined emotion, confirmation, and the refined treatment.

7. The system of claim 1, further comprising a third party data source providing third party information to said computer;
wherein determining an emotion associated with said image is based at least in part on the third-party information.

8. The system of claim 7, further comprising software executing on said computer for training a machine learning algorithm based on at least one of the determined emotion, confirmation, and the third party information.

9. The system of claim 7, wherein the third party data source provides information regarding at least one of weather at location of patient, potential stressors such as crime rate, pollution, traffic, and psycho economics such as stock price, inflation rate, employment rate and consumer index.

10. A system for managing treatment of a patient comprising:
a computer;
a database in data communication with said computer, said database having a plurality of prompts;
a user device in data communication with said computer for presenting a first prompt received from said computer to the patient;
a sensor associated with said user device for measuring a reaction of the patient in response to the prompt;
software executing on said computer for receiving the measurement and determining an emotion associated with said measurement; and
software executing on said computer querying the database based on the confirmed emotion to retrieve at least one additional prompts to be displayed to the patient to refine their treatment.

11. The system of claim 10, further comprising software executing on said computer for generating an emergency alert in response to the patient confirmation.

12. The system of claim 10, further comprising software executing on said computer for training a machine learning algorithm based on at least one of the determined emotion and confirmation.

13. The system of claim 10, further comprising a determined refinement to the treatment.

14. The system of claim 13, further comprising software executing on said computer for training a machine learning algorithm based on at least one of the determined emotion, confirmation, and the refined treatment.

15. The system of claim 10, further comprising a third party data source providing third party information to said computer;
wherein determining an emotion associated with said measurement is based at least in part on the third-party information.

16. The system of claim 15, further comprising software executing on said computer for training a machine learning algorithm based on at least one of the determined emotion, confirmation, and the third party information.

17. The system of claim 15, wherein the third party data source provides information regarding at least one of weather at location of patient, potential stressors such as crime rate, pollution, traffic, and psycho economics such as stock price, inflation rate, employment rate and consumer index.

18. The system of claim 10, wherein the sensor is a camera and the measurement is an image.

19. The system of claim 10 further comprising software executing on said user device for presenting the determined emotion to the patient; and software executing on said computer for receiving a confirmation as to whether the determined emotion is correct.
